(19) United States
(12) Patent Application Publication
(10) Pub. No.: US 2021/0017222 A1
(43) Pub. Date: Jan. 21, 2021

(54) SELECTIVE REDUCTION OF ANTIBODIES

(71) Applicant: AbbVie Inc., North Chicago, IL (US)

(72) Inventors: Anthony R Haight, Wadsworth, IL (US); Xiaoli Liao, Lincolnshire, IL (US)

(21) Appl. No.: 17/042,872

(22) PCT Filed: Mar. 29, 2019
(86) PCT No.: PCT/US19/24906

§ 371 Date: Sep. 28, 2020

Related U.S. Application Data
(60) Provisional application No. 62/650,112, filed on Mar. 29, 2018.

(51) Int. Cl. C07K 1/113; C07K 16/00; C07K 16/28; A61K 47/68

(52) U.S. Cl. CPC C07K 1/1133; C07K 16/00; A61K 47/6803; A61K 47/6849; C07K 16/2863

(57) ABSTRACT

The present disclosure provides methods for selectively reducing an antibody, comprising contacting the antibody with a reducing agent selected from the group consisting of 2-[2-(Diphenylphosphino)ethyl]pyridine, 3-(Diphenylphosphino)benzenesulfonic acid, 4-(Diphenylphosphino)benzoic acid, 2-(Diphenylphosphino)ethylamine, 3-(diphenylphosphino)propylamine, 3-(Diphenylphosphino)propionic acid, 2-(diisopropylphosphino)ethylamine, 2-(diphenylphosphino)benzoic acid, (2-hydroxyphenyl)diphenylphosphine, and salts thereof. The present disclosure further provides methods of producing antibody conjugates, such as antibody-drug conjugates.

### CROSS-REFERENCE TO RELATED APPLICATIONS

This application claims the benefit of U.S. Provisional Application No. 62/650,112, filed Mar. 29, 2018, the entire disclosure of which is incorporated by reference herein in its entirety.

### FIELD OF THE INVENTION

The present disclosure relates to a process for the site-specific reduction of antibodies and processes for the preparation of antibody conjugates (including antibody-drug conjugates).

### BACKGROUND

Antibody-drug conjugates (ADCs) are an emerging class of potent therapeutic agents, which have recently demonstrated remarkable clinical benefit. ADCs are comprised of at least one therapeutic or cytotoxic agent attached to an antibody via a stable linker. Putatively, by a series of events, ADCs may destroy cells possessing an over-expression of cell-surface proteins. In oncology, for example, ADCs combine the antigen-driven targeting properties of monoclonal antibodies with the potent anti-tumor effects of cytotoxic agents. For example, in 2011, ADCETRIS® (an anti-CDD30-MMAE ADC) gained regulatory approval for the treatment of refractory Hodgkin lymphoma and systemic anaplastic lymphoma.

Many ADCs rely on chemical conjugation methods to link therapeutic drugs to lysines or cysteines on the native antibody. These conjugates are heterogeneous mixtures with a varying number of drugs attached at multiple positions on the antibody. Studies have demonstrated deleterious effects of a high drug loaded ADCs (Liu et al. (2010) Analy. Chem. 82:5219). These deleterious effects of higher levels of conjugation include increased propensity towards aggregate formation (King et al. (2002) J Med. Chem. 45:4336; Hollander et al. (2008) Bioconjugate Chem 19:358; Burke et al. (2009) Bioconjugate Chem 20: 1242; and Zhao et al. (2011) J Med. Chem. 54:3606).

Controlling the drug load of an ADC by conjugation to a cysteine has been attempted using various methods, including: i) engineering into the antibody unpaired cysteines or unnatural amino acids that are amenable to conjugation, ii) utilizing transglutaminase (TG), sortase, or Formylglycine generating enzyme (FGE) while incorporating corresponding tags, and iii) post-translational glycan modification or N-terminal modification. Reviews of methods for site-specific drug conjugation to antibodies can be found, for example, in mAbs 2014, 6, 34-45; mAbs, 2014, 6, 46-53; and Biotechnol. Adv. 2015, 33, 775-784. These strategies can be used to successfully generate controlled loading site-specific conjugations but may require screening for appropriate positions on the antibody to achieve the desired reactivity, re-optimization of cell culture conditions (for non-natural amino acid incorporation), extensive enzymatic treatments, or harsh oxidation conditions.

For example, one technique for site-specific conjugation, described in WO2006/034488, successfully controls drug-loading and the site of drug incorporation by replacing one or more amino acids of a parent antibody with non-cross-linked, highly reactive cysteine amino acids at positions showing high thiol reactivity. However, such methods require screening for appropriate positions on the antibody to achieve the desired reactivity.

Moreover, engineered cysteine residues in suitable positions of the mutated antibody are often capped by other thiols (e.g., cysteine or glutathione) to form disulfides, and must therefore be uncapped before cytotoxic or therapeutic moieties can be attached. Partial reduction and oxidation may be used to uncap the newly introduced cysteines and reform the inter-chain disulfide bond. However, such re-oxidation steps can have unintended consequences. For example, the re-oxidation process can cause disulfide scrambling (that is, disulfide bonds between cysteines that were not originally paired in the unreduced antibody), and otherwise add complexity to the ADC preparation process.

Further, when frequently used reductants such as Tris(2-carboxyethyl)phosphine (TCEP) are used to reduce antibodies (such as cysteine engineered antibodies), reduction of the inter-chain disulfide bonds at the Fc region can occur, leading to a high drug-antibody ratio (DAR) and undesirable toxicity of the resulting ADC.

Accordingly, there remains a need for new methods for the selective reduction of cysteine-engineered antibodies and compositions that can provide optimal drug loading and selective enrichment of ADCs with desirable drug loading species while significantly reducing the need for downstream chromatographic purification.

### SUMMARY OF THE INVENTION

In embodiments, the present disclosure provides a method for selectively reducing a protein, where the method involves contacting the protein with a reducing agent. In embodiments, the reducing agent is selected from the group consisting of 2-[2-(Diphenylphosphino)ethyl]pyridine, 3-(Diphenylphosphino)benzenesulfonic acid, 4-(Diphenylphosphino)benzoic acid, 2-(Diphenylphosphino)ethylamine, 3-(diphenylphosphino)propylamine, 3-(Diphenylphosphino)propionic acid, 2-(diisopropylphosphino)ethylamine, 2-(diphenylphosphino)benzoic acid, (2-hydroxyphenyl)diphenylphosphine, and salts thereof. In embodiments, the protein is an antibody. In embodiments, the antibody includes at least one cysteine residue that has either been replaced by another amino acid, such as serine or alanine.

In embodiments, the present disclosure provides a method of preparing an antibody conjugate, where the method comprises contacting an antibody with a reducing agent. In embodiments, the reducing agent is selected from the group consisting of 2-[2-(Diphenylphosphino)ethyl]pyridine, 3-(Diphenylphosphino)benzenesulfonic acid, 4-(Diphenylphosphino)benzoic acid, 2-(Diphenylphosphino)ethylamine, 3-(diphenylphosphino)propylamine, 3-(Diphenylphosphino)propionic acid, 2-(diisopropylphosphino)ethylamine, 2-(diphenylphosphino)benzoic acid, (2-hydroxyphenyl)diphenylphosphine, and salts thereof. In embodiments, the method further comprises combining the antibody with a drug-linker compound under conditions sufficient to form an antibody-drug conjugate.

In embodiments, the present disclosure provides a method for the preparation of an antibody conjugate, where the method involves providing an antibody in which a cysteine residue of the antibody has been replaced by another amino acid in the C214 position of the light chain or the C220 position in the heavy chain, according to the Eu system of Kabat. In embodiments, the method comprises contacting the antibody with a reducing agent selected from the group consisting of 2-[2-(Diphenylphosphino)ethyl]pyridine, 3-(Diphenylphosphino)benzenesulfonic acid, 4-(Diphenylphosphino)benzoic acid, 2-(Diphenylphosphino)ethylamine, 3-(diphenylphosphino)propylamine, 3-(Diphenylphosphino)propionic acid, 2-(diisopropylphosphino)ethylamine, 2-(diphenylphosphino)benzoic acid, (2-hydroxyphenyl)diphenylphosphine, and salts thereof.

### BRIEF DESCRIPTION OF THE FIGURES

FIGS. 1A and B shows a general overview of the site-specific reduction and conjugation strategies of the present disclosure using antibodies comprising unpaired cysteines at HC220 (FIG. 1A) or antibodies comprising unpaired cysteines at LC214 (FIG. 1B).

FIG. 2A shows a specific overview describing the preparation of DAR2, DAR4, and dual drug-linker ADCs from a C6v1 antibody (LC214; an antibody in which the cysteine residing at amino acid residue 214 of the light chain has either been deleted or replaced with a non-cysteine amino acid) through selective reduction according to the present disclosure. FIG. 2B shows preparation of DAR2, DAR4, and dual drug-linker ADCs from a C6v2 antibody (HC220; an antibody in which the cysteine residing at amino acid residue 220 of the heavy chain has either been deleted or replaced with a non-cysteine amino acid) through selective reduction according to the present disclosure. In each instance, use of the reductants in conjunction with the C6v1 or C6v2 antibodies results in the selective reduction of the two “unpaired cysteines” in the fab region of the heavy chain (HC220) for C6v1 and the light chain (LC214) for C6v2, which facilitates generation of antibody-drug conjugates with a DAR of 2, 4, or dual drug-linker ADCs.

FIG. 3A-B shows bar graphs representing (A) the DAR profile from HIC and (B) the conjugation profile from RP-HPLC from reactions of Cetuximab-C6v1 with three reductants according to the present disclosure (specifically, the reductants 3-(Diphenylphosphino)benzenesulfonic acid sodium salt, 4-(Diphenylphosphino)benzoic acid, and 2-(Diphenylphosphino)ethylamine, as illustrated below), followed by MC-VC-MMAE conjugation, as described in Example 1. As shown in FIG. 3A, the selective reduction process according to the present disclosure yielded not only a high level of desired DAR2 or D2 species by HIC, but also a very low level of high drug loading species (DAR4/DAR6, or D4/D6), indicating minimal reduction of disulfide bonds in the Fc region. FIG. 3B shows the analysis by RP-HPLC, where H0 indicates heavy chain with no toxin conjugated, H1 indicates heavy chain with one toxin conjugated, and H2/H3 indicates heavy chain with 2 or 3 toxins conjugated. As shown in FIG. 3B, the data showed >90% of H1, with minimal reduction of the interchain disulfide bonds at the Fc region (as indicated by the low level of H2/H3).

FIG. 4A-B shows bar graphs representing (A) the DAR profile from HIC and (B) the conjugation profile from RP-HPLC of the reactions of Cetuximab-C6v2 with three reductants according to the present disclosure (specifically, reductants 4-(Diphenylphosphino)benzoic acid, 2-(Diphenylphosphino)ethylamine, and 3-(diphenylphosphino)propylamine, illustrated below), followed by MC-VC-MMAE as described in Example 1. The reaction was carried out at 0° C. with a buffer of pH 7.2 except where designated with a *, which indicates a buffer of pH 8. As shown in FIG. 4A, selective reduction using these reactants not only resulted in >85% of DAR2 for C6v2, but also produced a low level of the high drug loading species (D4/D6). As shown in FIG. 4B, analysis by RP-HPLC reveals >93% of L1 (i.e., one toxin conjugation at the LC214 position), with the undesirable H1/H2 species (heavy chain with one or two toxins conjugated) controlled to low levels. This suggests that undesirable reduction of disulfide bonds in the Fc region is controlled to low levels.

FIG. 5A-B shows bar graphs that demonstrate the pH impact on (A) the DAR profile from HIC, and (B) the conjugation profile from RP-HPLC for reactions of Cetuximab-C6v2 with 3-(diphenylphosphino)propylamine and 3-(Diphenylphosphino)propionic acid, and for reduction with 3-(Diphenylphosphino)propionic acid of a C6v2 antibody to a different target (Target 4) (designated as # in FIG. 5A), in both cases followed by MC-VC-MMAE conjugation, as described in Example 1. The reactions were carried out at 0° C. with a buffer of pH 7.2 except where designated with a *, which indicates a buffer of pH 8.

FIG. 6A-B shows bar graphs that illustrate the DAR profile from HIC of the reactions of (A) reductant 2-(Diphenylphosphino)ethylamine on three C6v1 antibodies directed to different targets, and (B) reductant 4-(Diphenylphosphino)benzoic acid on seven C6v2 antibodies directed to different targets, followed by MC-VC-MMAE conjugation, as described in Example 2. The reactions were carried out with a buffer of pH 7.2 at 0° C. overnight except where designated with a *, which indicates a buffer of pH 8 at 4° C. overnight. As shown in FIG. 6A, the three C6v1 mutant antibodies (Cetuximab-C6v1, Trastuzumab-C6v1, and Target 1-C6v1) tested with 2-(Diphenylphosphino)ethylamine (reductant 5) had similar HIC profiles with ≥85%, D2, and ≤5%, of high drug loading (>D2) species by HIC. As shown in FIG. 6B, for seven C6v2 antibodies (i.e., Cetuximab, Trastuzumab, Target 2, Target 3, Target 4, Target 5, and Target 6) tested with 4-(Diphenylphosphino)benzoic acid (reductant 4), all seven antibodies showed ≥85%, D2 and ≤4%, of high drug loading (>D2) species by HIC, despite their difference in sequence and the percentage (8-35%) of capped cysteine at position LC214.

FIG. 7 shows nineteen reductants evaluated in the Examples of the present disclosure.

### DETAILED DESCRIPTION OF THE INVENTION

In embodiments, the present disclosure provides a process for the selective reduction of proteins, which can be applied to the preparation of site-specific ADCs. In embodiments, the method utilizes mild phosphine-based reductants to reduce unpaired cysteines in the Fab region of the heavy chain or the light chain of an antibody, while keeping the inter-chain disulfide bonds in the Fc region intact. Following reduction according to the present disclosure, a drug-linker compound can then be directly added to generate site-specific ADCs with a drug-to-antibody ratio of about 2.

When reductants such as Tris(2-carboxyethyl)phosphine (TCEP) are used to reduce antibodies (such as cysteine engineered antibodies), reduction of the inter-chain disulfide bonds at the Fc region is observed, reflected in high drug loading species by HIC and PLRP-S HPLC. Moreover, full reduction by TCEP and partial re-oxidation by dehydroascorbic acid non-selectively yields a mixture of drug loading species when applied to C6v1 antibodies (i.e., an engineered antibody in which the C214 residue of the light chain is substituted with another residue). In contrast, reduction of cysteine engineered antibodies with the reductants according in the instant disclosure is highly specific for species with a drug loading of 2, selectively yielding DAR2 species (in embodiments, greater than about 70% DAR2, or greater than about 80% DAR2, or greater than about 85% DAR2) while keeping higher drug loading species (e.g., DAR4, DAR6) to a minimum (i.e., less than about 20%, or less than about 10%, or less than about 5%).

Reduction of wild-type antibodies with the reductants according to the instant disclosure is more specific for species with a drug loading of 4 than the commonly-used TCEP, and thus can enhance the yield of the DAR4 species generated from reducing a wild-type antibody.

Moreover, the selective reduction method according to the present disclosure offers the ability to control drug loading and conjugation site using a mild, one-pot process. Conventional conjugation procedures for cysteine-engineered antibody ADCs involve, for example, full reduction of the cysteine-engineered antibody, followed by partial re-oxidation of the interchain disulfide bonds, before the antibody can be conjugated to a drug/toxin. See, e.g., WO2006/034488. Such reoxidation steps can have unintended consequences, such as disulfide scrambling, and add potential complexity to ADC preparation. The use of a phosphine-based reductant according to the present disclosure is compatible with one-pot reduction and conjugation processing and does not necessarily require additional steps to remove excess reductant prior to introduction of the drug-linker. In embodiments, the process according to the present disclosure involves a direct addition of a therapeutic or cytotoxic agent for conjugation in one pot without additional steps between reduction and conjugation. The process according to the present disclosure increases overall yield and improves the operational efficiency and simplicity of the ADC preparation process, as the need for chromatographic purification is reduced due to the high purity and site-specificity of the ADCs generated according to the present disclosure.

In embodiments, the present disclosure relates to a process for the selective reduction of a protein, comprising contacting the protein with a reducing agent selected from the group consisting of 2-[2-(Diphenylphosphino)ethyl]pyridine, 3-(Diphenylphosphino)benzenesulfonic acid, 4-(Diphenylphosphino)benzoic acid, 2-(Diphenylphosphino)ethylamine, 3-(diphenylphosphino)propylamine, 3-(Diphenylphosphino)propionic acid, 2-(diisopropylphosphino)ethylamine, 2-(diphenylphosphino)benzoic acid, (2-hydroxyphenyl)diphenylphosphine, and salts thereof. In embodiments, the protein is an antibody.

In embodiments, the antibody is a wild-type antibody. In embodiments, the antibody is an engineered antibody which has at least one cysteine residue that has been replaced by another amino acid. For example, in embodiments, the antibody is an engineered antibody in which the C214 residue (according to the Eu system of Kabat) of the light chain is substituted with another residue (referred to as “C6v1”). In other embodiments, the antibody is an engineered antibody in which the C220 residue (according to the Eu system of Kabat) of the heavy chain is substituted by another residue (referred to as “C6v2”). In embodiments, the substituted residue is an L-amino acid. In embodiments, the substituted residue may be alanine or serine. Site-specific antibodies comprising the engineered C214 or C220 residues are described in, for example, U.S. Patent Application Publication No. 2016/0176964, U.S. Patent Application Publication No. 2008/0305044, and Protein Engineering, Design & Selection 2006, 19, 299-307, the entire disclosures of which are incorporated by reference herein in their entireties. These mutant antibodies require no additional screening for sites of mutation and are readily generated based on different antibody backbones.

In embodiments, the antibody is an engineered antibody in which at least one cysteine residue has been deleted.

The terms “Kabat numbering”, “Kabat definitions”, and “Kabat labeling” are used interchangeably, and refer to a system of numbering amino acid residues which are more variable (i.e., hypervariable) than other amino acid residues in the heavy and light chain variable regions of an antibody or binding protein, or an antigen binding portion thereof (Kabat et al. (1971) Ann. NY Acad. Sci.190:382-391 and, Kabat et al. (1991) Sequences of Proteins of Immunological Interest, Fifth Edition, U.S. Department of Health and Human Services, NIH Publication No.91-3242). “Eu numbering” or “Eu system” refers to the Eu index as described in (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242).

The terms “drug load” or “drug loading” refer to the molar ratio of drug molecules per antibody in an individual ADC molecule. In embodiments, the drug loading may comprise from 1-8 drug molecules, from 2-4 drug molecules, from 1-3 drug molecules, or from 2-3 drug molecules. In embodiments, the drug loading may comprise 1 drug molecule, 2 drug molecules, 3 drug molecules, 4 drug molecules, 5 drug molecules, 6 drug molecules, 7 drug molecules, or 8 drug molecules. In embodiments, the general formula of an ADC molecule may be represented as A(-L-D)ₙ, wherein A is the antibody, L is the linker, D is the drug, and n is the number of drug molecules linked to the antibody. In embodiments, n is from 1-8, or from 1-4, or from 2-4, or from 1-3, or from 2-3. In embodiments, n is 1, 2, 3, 4, 5, 6, 7, or 8. As used herein, a particular drug-loading species is referred to as “DAR-n” or “D-n”, where n is the drug loading of the species (for example, a species with 2 drug molecules attached to the antibody would be DAR2 or D2, a species with 4 drug molecules attached to the antibody would be DAR4 or D4, and so forth).

The terms “drug-to-antibody ratio” or “DAR” of a given composition refer to the weighted average molar ratio of drug molecules per antibody in a population of at least two ADC molecules. Despite the conjugate specificity provided by the methods of the present disclosure, a given population of ADCs may comprise ADC molecules having different drug loadings (e.g., ranging from 1 to 8 in the case of an IgG1 antibody). That is, following conjugation, ADC compositions of the invention may comprise a mixture of ADCs with different drug loadings. Hence, DAR represents the weighted average of drug loadings for the ADC population as a whole (i.e., all the ADC molecules taken together).

In embodiments, the ADCs of the present disclosure have a DAR of about 2, or a DAR of about 4. In this context, the term “about” means an amount within ±7.5% of the actual value; i.e., “about 2” means 1.85, 1.86, 1.87, 1.88, 1.89, 1.90, 1.91, 1.92, 1.93, 1.94, 1.95, 1.96, 1.97, 1.98, 1.99, 2.00, 2.01, 2.02, 2.03, 2.04, 2.05, 2.06, 2.07, 2.08, 2.09, 2.10, 2.11, 2.12, 2.13, 2.14, 2.15, and any number or range between them.

The terms “engineered antibody,” “engineered construct”, and “site-specific antibody” refer to antibodies wherein at least one amino acid in either the heavy or light chain is deleted, altered, or substituted (in embodiments with another amino acid) to provide at least one unpaired cysteine. Similarly, an “engineered conjugate” or “site-specific conjugate” refers to an antibody-drug conjugate comprising an engineered antibody and at least one cytotoxin conjugated to the unpaired cysteine(s). In certain embodiments, the unpaired cysteine residue will comprise an unpaired interchain cysteine residue. The engineered antibody can be of various isotypes, for example, IgG1, IgG2, IgG3, or IgG4.

In embodiments, the substituted or deleted cysteine residue is on the light chain (either kappa or lambda) thereby leaving the unpaired cysteine on the heavy chain. In other embodiments the substituted cysteine residue is on the heavy chain, leaving the unpaired cysteine on the light chain. Deletion or substitution of one cysteine in either the light or heavy chain of an intact antibody results in an engineered antibody having two unpaired cysteine residues. Thus, in embodiments, C220 on the heavy chain is substituted with another amino acid, such as alanine or serine, to provide the desired unpaired cysteine in the light chain. In other embodiments, C214 in the light chain is substituted with another amino acid, such as serine or alanine, to provide the desired unpaired cysteine in the heavy chain.

In embodiments, the methods according to the present disclosure may be used to enhance the selectivity in reducing cysteine residues in a wild-type antibody. In embodiments, the reduced antibodies may be used to generate antibody-drug conjugates. In embodiments, the methods of the present disclosure allow for enrichment of a particular species, such as a DAR4 species, to a greater extent than what is attainable using TCEP as a reagent.

In embodiments, the methods according to the present disclosure may be used to selectively reduce the two unpaired cysteines in the fab region of an engineered antibody comprising engineered C214 or C220 residues while keeping the interchain disulfide bonds in the Fc region intact. The reduced antibodies may then be used to generate site-specific antibody-drug conjugates. In embodiments, the reduced antibodies may be used to generate site-specific antibody-drug conjugates by reaction of a thiol reactive agent, such as a maleimide-containing drug-linker, with the unpaired thiols.

In embodiments, the present disclosure provides a method for selectively reducing an antibody, wherein the antibody is an engineered antibody in which the C214 residue (according to the Eu system of Kabat) of the light chain is substituted with another residue, where the method involves contacting the antibody with a reducing agent selected from the group consisting of: 2-[2-(Diphenylphosphino)ethyl]pyridine, 3-(Diphenylphosphino)benzenesulfonic acid, 4-(Diphenylphosphino)benzoic acid, 2-(Diphenylphosphino)ethylamine, 3-(diphenylphosphino)propylamine, 2-(diphenylphosphino)benzoic acid, (2-hydroxyphenyl)diphenylphosphine, and salts thereof. In embodiments, the reducing agent is selected from the group consisting of 3-(Diphenylphosphino)benzenesulfonic acid, 4-(Diphenylphosphino)benzoic acid, 2-(Diphenylphosphino)ethylamine, 3-(diphenylphosphino)propylamine, 2-(diphenylphosphino)benzoic acid, (2-hydroxyphenyl)diphenylphosphine, and salts thereof. In embodiments, the reducing agent is selected from the group consisting of 3-(Diphenylphosphino)benzenesulfonic acid, 4-(Diphenylphosphino)benzoic acid, 2-(Diphenylphosphino)ethylamine, 2-(diphenylphosphino)benzoic acid, and salts thereof.

In embodiments, the present disclosure provides a method for selectively reducing an antibody, wherein the antibody is an engineered antibody in which the C220 residue (according to the Eu system of Kabat) of the heavy chain is substituted by another residue, where the method comprises contacting the antibody with a reducing agent selected from the group consisting of 2-[2-(Diphenylphosphino)ethyl]pyridine, 3-(Diphenylphosphino)benzenesulfonic acid, 4-(Diphenylphosphino)benzoic acid, 2-(Diphenylphosphino)ethylamine, 3-(diphenylphosphino)propylamine, 3-(Diphenylphosphino)propionic acid, 2-(diisopropylphosphino)ethylamine, and salts thereof. In embodiments, the reducing agent is selected from the group consisting of 3-(Diphenylphosphino)benzenesulfonic acid, 4-(Diphenylphosphino)benzoic acid, 2-(Diphenylphosphino)ethylamine, 3-(diphenylphosphino)propylamine, 3-(Diphenylphosphino)propionic acid, and salts thereof. In embodiments, the reducing agent is selected from the group consisting of 4-(Diphenylphosphino)benzoic acid, 2-(Diphenylphosphino)ethylamine, 3-(diphenylphosphino)propylamine, 3-(Diphenylphosphino)propionic acid, and salts thereof. In embodiments, the reducing agent is selected from the group consisting of 4-(Diphenylphosphino)benzoic acid, 2-(Diphenylphosphino)ethylamine, 3-(diphenylphosphino)propylamine, and salts thereof.

In embodiments, the reducing agent is present in an amount of about two molar equivalents per molar amount of engineered cysteine. In embodiments, the reducing agent is present in an amount of at least two molar equivalents per molar amount of engineered cysteine, such as from about 2 to about 10 molar equivalents, or from about 2 to about 8 molar equivalents, or from about 2 to about 6 molar equivalents, or from about 2 to about 4 molar equivalents per molar amount of engineered cysteine.

In embodiments, after the antibody is contacted with the reducing agent, substantially all of the inter-chain disulfide bonds present in the Fc region of the antibody are retained after the reduction reaction. That is, the reducing agents selectively reduce the two unpaired cysteines in the Fab region of the antibody while keeping the interchain disulfide bonds in the Fc region intact.

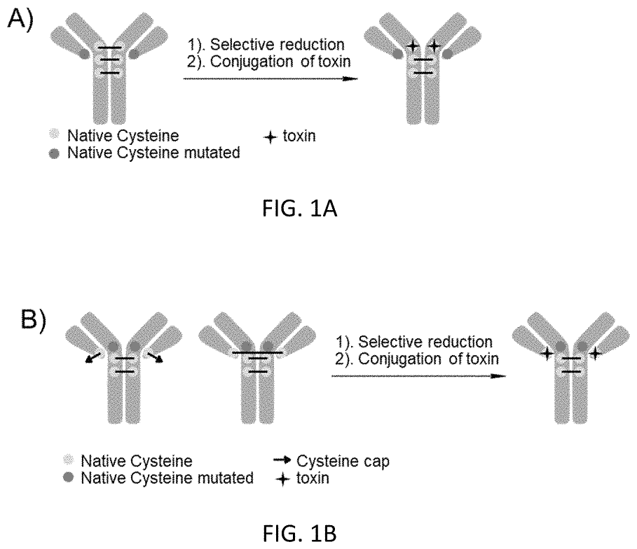

Following reduction with the phosphine-based reductants according to the present disclosure, the antibody can be used to generate site-specific antibody conjugates, such as creation of site-specific antibody-drug conjugates by reaction of a thiol reactive agent (such as a maleimide containing drug-linker compound) with the free thiols, as described in FIGS. 1A and B.

The term “antibody-drug-conjugate” or “ADC” refers to a binding protein, such as an antibody or antigen binding fragment thereof, chemically linked to one or more chemical drug(s) (also referred to herein as agent(s)) that may optionally be therapeutic or cytotoxic agents. In a preferred embodiment, an ADC includes an antibody, a cytotoxic or therapeutic drug, and a linker that enables attachment or conjugation of the drug to the antibody. For example, in embodiments, the antibody may be conjugated to a therapeutic moiety, a radiopharmaceutical, a radiolabel, an enzyme, a fluorescent label, a luminescent label, a bioluminescent label, a magnetic label, biotin, a fluorescent label, or a hydrophilic polymer via a cleavable or non-cleavable linker. Non-limiting examples of drugs that may be included in the ADCs include mitotic inhibitors, antitumor antibiotics, immunomodulating agents, vectors for gene therapy, alkylating agents, antiangiogenic agents, antimetabolites, boron-containing agents, chemoprotective agents, hormones, antihormone agents, corticosteroids, photoactive therapeutic agents, oligonucleotides, radionuclide agents, topoisomerase inhibitors, tyrosine kinase inhibitors, and radiosensitizers.

Generally speaking, the agents set forth above, as well as other agents, may be attached to the reduced antibodies of the present disclosure using techniques that are known in the art. See, for example and without limitation, Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, In Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62: 119-58 (1982), each of which is hereby incorporated by reference in its entirety.

In embodiments, the present disclosure provides methods of preparing an antibody drug conjugate. In embodiments, the method involves providing an antibody, and contacting the antibody with a reductant selected from the group consisting of 2-[2-(Diphenylphosphino)ethyl]pyridine, 3-(Diphenylphosphino)benzenesulfonic acid, 4-(Diphenylphosphino)benzoic acid, 2-(Diphenylphosphino)ethylamine, 3-(diphenylphosphino)propylamine, 3-(Diphenylphosphino)propionic acid, 2-(diisopropylphosphino)ethylamine, 2-(diphenylphosphino)benzoic acid, (2-hydroxyphenyl)diphenylphosphine, and salts thereof. In embodiments, the antibody may be an engineered antibody in which the C214 residue of the light chain is substituted by another residue (C6v1) or an engineered antibody in which the C220 residue of the heavy chain is substituted by another residue (C6v2). In embodiments, the method further involves contacting the antibody with a thiol reactive agent, such as a drug-linker compound, under conditions sufficient to form an antibody-drug conjugate.

In embodiments, the average drug load of the resulting antibody-drug conjugate is about 2 drug moieties per antibody. For example, in embodiments, more than about 70% of the antibody-drug conjugates produced according to the process have a drug loading of 2. In embodiments, more than about 75% of the antibody-drug conjugates produced according to the process have a drug loading of 2. In embodiments, more than about 80% of the antibody-drug conjugate produced according to the process of the present disclosure have a drug loading of 2. In embodiments, more than about 85% of the antibody-drug conjugates produced according to the process of the present disclosure have a drug loading of 2. In embodiments, more than about 90% of the antibody-drug conjugates produced according to the process of the present disclosure have a drug loading of 2.

In embodiments, the present disclosure provides methods of preparing an antibody drug conjugate. In embodiments, the method involves providing an antibody, wherein the antibody is an engineered antibody in which the C214 residue (according to the Eu system of Kabat) of the light chain is substituted with another residue, and contacting the antibody with a reductant selected from the group consisting of 2-[2-(Diphenylphosphino)ethyl]pyridine, 3-(Diphenylphosphino)benzenesulfonic acid, 4-(Diphenylphosphino)benzoic acid, 2-(Diphenylphosphino)ethylamine, 3-(diphenylphosphino)propylamine, 2-(diphenylphosphino)benzoic acid, (2-hydroxyphenyl)diphenylphosphine, and salts thereof. In embodiments, the reducing agent is selected from the group consisting of 3-(Diphenylphosphino)benzenesulfonic acid, 4-(Diphenylphosphino)benzoic acid, 2-(Diphenylphosphino)ethylamine, 3-(diphenylphosphino)propylamine, 2-(diphenylphosphino)benzoic acid, (2-hydroxyphenyl)diphenylphosphine, and salts thereof. In embodiments, the reducing agent is selected from the group consisting of 3-(Diphenylphosphino)benzenesulfonic acid, 4-(Diphenylphosphino)benzoic acid, 2-(Diphenylphosphino)ethylamine, 2-(diphenylphosphino)benzoic acid, and salts thereof. In embodiments, the method further involves contacting the antibody with a thiol reactive agent, such as a drug-linker compound, under conditions sufficient to form an antibody-drug conjugate. In embodiments, the average drug load of the resulting antibody-drug conjugate is about 2 drug moieties per antibody. For example, in embodiments, more than about 70% of the antibody-drug conjugates produced according to the process have a drug loading of 2. In embodiments, more than about 75% of the antibody-drug conjugates produced according to the process have a drug loading of 2. In embodiments, more than about 80% of the antibody-drug conjugate produced according to the process of the present disclosure have a drug loading of 2. In embodiments, more than about 85% of the antibody-drug conjugates produced according to the process of the present disclosure have a drug loading of 2. In embodiments, more than about 90% of the antibody-drug conjugates produced according to the process of the present disclosure have a drug loading of 2.

In embodiments, the present disclosure provides methods of producing an antibody-drug conjugate, where the method comprises providing an antibody, wherein the antibody is an engineered antibody in which the C220 residue (according to the Eu system of Kabat) of the heavy chain is substituted (i.e., C6v2), and contacting the antibody with a reducing agent selected from the group consisting of 2-[2-(Diphenylphosphino)ethyl]pyridine, 3-(Diphenylphosphino)benzenesulfonic acid, 4-(Diphenylphosphino)benzoic acid, 2-(Diphenylphosphino)ethylamine, 3-(diphenylphosphino)propylamine, 3-(Diphenylphosphino)propionic acid, 2-(diisopropylphosphino)ethylamine, and salts thereof. In embodiments, the reducing agent is selected from the group consisting of 3-(Diphenylphosphino)benzenesulfonic acid, 4-(Diphenylphosphino)benzoic acid, 2-(Diphenylphosphino)ethylamine, 3-(diphenylphosphino)propylamine, 3-(Diphenylphosphino)propionic acid, and salts thereof. In embodiments, the reducing agent is selected from the group consisting of 4-(Diphenylphosphino)benzoic acid, 2-(Diphenylphosphino)ethylamine, 3-(diphenylphosphino)propylamine, 3-(Diphenylphosphino)propionic acid, and salts thereof. In embodiments, the reducing agent is selected from the group consisting of 4-(Diphenylphosphino)benzoic acid, 2-(Diphenylphosphino)ethylamine, 3-(diphenylphosphino)propylamine, and salts thereof. The antibody may then be contacted with a thiol reactive agent, such as a drug-linker compound, under conditions sufficient to form an antibody-drug conjugate. In embodiments, the average drug load of the resulting antibody-drug conjugate is about 2 drug moieties per antibody. For example, in embodiments, more than about 70% of the antibody-drug conjugates produced according to the process have a drug loading of 2. In embodiments, more than about 75% of the antibody-drug conjugates produced according to the process have a drug loading of 2. In embodiments, more than about 80% of the antibody-drug conjugates produced according to the process have a drug loading of 2. In embodiments, more than about 85% of the antibody-drug conjugates produced according to the process have a drug loading of 2. In embodiments, more than about 90% of the antibody-drug conjugates produced according to the process have a drug loading of 2.

As discussed above, an antibody drug conjugate may be prepared by contacting the reduced antibody with a thiol reactive agent, such as a drug-linker compound, under conditions sufficient to form an antibody drug conjugate. In embodiments, the thiol reactive agent may be a therapeutic moiety (such as a toxin or drug) that is conjugated to the antibody via a cleavable or non-cleavable linker to form the antibody-drug conjugate. In embodiments, the cleavable or non-cleavable linker has a functional group that can react with the thiol group of the uncapped engineered cysteine. For example, in embodiments, the functional group may be a maleimide group or a haloacetyl group. Cleavable linkers include, for example, a valine-citrulline (vc) linker or a valine-alanine (va) linker moiety.

In embodiments, the present disclosure provides an antibody-drug conjugate prepared according to the methods discussed above. In embodiments, the antibody is a C6v1 or C6v2 cysteine engineered antibody. In embodiments, the average DAR of the antibody-drug conjugates produced according to the present disclosure is about 2. In embodiments, more than about 70% of the antibody-drug conjugates produced according to the process have a drug loading of 2. In embodiments, more than about 75% of the antibody-drug conjugates produced according to the process have a drug loading of 2. In embodiments, more than about 80% of the antibody-drug conjugates produced according to the process have a drug loading of 2. In embodiments, more than about 85% of the antibody-drug conjugates produced according to the process have a drug loading of 2. In embodiments, more than about 90% of the antibody-drug conjugates produced according to the process have a drug loading of 2.

In embodiments, the present disclosure provides a composition comprising antibody-drug conjugates, wherein the average DAR of the composition is 2. In embodiments, more than about 70% of the antibody-drug conjugates in the composition have a drug loading of 2. In embodiments, more than about 75% of the antibody-drug conjugates in the composition have a drug loading of 2. In embodiments, more than about 80% of the antibody-drug conjugates in the composition have a drug loading of 2. In embodiments, more than about 85% of the antibody-drug conjugates in the composition have a drug loading of 2. In embodiments, more than about 90% of the antibody-drug conjugates in the composition have a drug loading of 2. In embodiments, the composition comprises less than about 20% of higher drug loading species (i.e., species with a higher drug loading than 2, e.g., DAR4, DAR6), or less than about 15% higher drug loading species, or less than about 10% higher drug loading species, or less than about 5% higher drug loading species. For example, in embodiments, the present disclosure provides a composition comprising ADCs, wherein the composition comprises at least about 70% DAR2 species and less than about 20% of higher drug loading species (e.g., DAR4/DAR6), or at least about 75% DAR2 species and less than about 15% higher DAR species, or at least about 80% DAR2 species and less than about 10% higher DAR species, or at least about 85% DAR2 species and less than about 5% higher DAR species.

In embodiments, the methods of the present disclosure may be further used to prepare antibody conjugates with a drug loading of 4. For example, in embodiments, the present disclosure provides methods for preparing D2 antibody drug conjugates, comprising providing an antibody, and contacting the antibody with a reductant selected from the group consisting of 2-[2-(Diphenylphosphino)ethyl]pyridine, 3-(Diphenylphosphino)benzenesulfonic acid, 4-(Diphenylphosphino)benzoic acid, 2-(Diphenylphosphino)ethylamine, 3-(diphenylphosphino)propylamine, 3-(Diphenylphosphino)propionic acid, 2-(diisopropylphosphino)ethylamine, 2-(diphenylphosphino)benzoic acid, (2-hydroxyphenyl)diphenylphosphine, and salts thereof. In embodiments, the antibody may be a wild-type antibody, and the process according to the present disclosure may provide a DAR4 yield greater than what is achieved using TCEP. For example, in embodiments, more than 50% of the antibody-drug conjugates produced according to the process will have a drug loading of 4, or more than 55% of the antibody-drug conjugates will have a drug loading of 4. In embodiments, the antibody may be an engineered antibody in which the C214 residue of the light chain is substituted by another residue or an engineered antibody in which the C220 residue of the heavy chain is substituted by another residue. The two cysteines at HC220 (C6v1) or LC214 (C6v2) that are revealed by selective reduction according to the present disclosure may then be capped (by, for example, contacting the antibody with N-ethylmaleimide or another maleimide containing small molecule). The method then further involves reducing the two interchain disulfide bonds of the Fc region with TCEP, thus enabling subsequent conjugation of four drug/toxins to the antibody to generate D4 species. In embodiments, more than about 70% of the antibody-drug conjugates produced according to the process have a drug loading of 4. In embodiments, more than about 75% of the antibody-drug conjugates have a drug loading of 4. In embodiments, more than about 80% of the antibody-drug conjugates have a drug loading of 4. In embodiments, more than about 85% of the antibody drug-conjugates have a drug loading of 4. In embodiments, more than about 90% of the antibody-drug conjugates have a drug loading of 4.

In embodiments, the methods of the present disclosure may be used to prepare antibody conjugates with a DAR of 4 using an engineered antibody in which the C214 residue (according to the Eu system of Kabat) of the light chain is substituted with another residue, where the method involves contacting the antibody with a reducing agent selected from the group consisting of: 2-[2-(Diphenylphosphino)ethyl]pyridine, 3-(Diphenylphosphino)benzenesulfonic acid, 4-(Diphenylphosphino)benzoic acid, 2-(Diphenylphosphino)ethylamine, 3-(diphenylphosphino)propylamine, 2-(diphenylphosphino)benzoic acid, (2-hydroxyphenyl)diphenylphosphine, and salts thereof. In embodiments, the reducing agent is selected from the group consisting of 3-(Diphenylphosphino)benzenesulfonic acid, 4-(Diphenylphosphino)benzoic acid, 2-(Diphenylphosphino)ethylamine, 3-(diphenylphosphino)propylamine, 2-(diphenylphosphino)benzoic acid, (2-hydroxyphenyl)diphenylphosphine, and salts thereof. In embodiments, the reducing agent is selected from the group consisting of 3-(Diphenylphosphino)benzenesulfonic acid, 4-(Diphenylphosphino)benzoic acid, 2-(Diphenylphosphino)ethylamine, 2-(diphenylphosphino)benzoic acid, and salts thereof. The two cysteines at HC220 (C6v1) that are revealed by the selective reduction process according to the present disclosure may then be capped (by, for example, contacting the antibody with N-ethylmaleimide or another maleimide containing small molecule). The method then further involves reducing the two interchain disulfide bonds of the Fc region with TCEP, thus enabling subsequent conjugation of four drug/toxins to the antibody to generate DAR4 species. In embodiments, more than about 70% of the antibody-drug conjugates produced according to the process have a drug loading of 4. In embodiments, more than about 75% of the antibody-drug conjugates have a drug loading of 4. In embodiments, more than about 80% of the antibody-drug conjugates have a drug loading of 4. In embodiments, more than about 85% of the antibody drug-conjugates have a drug loading of 4. In embodiments, more than about 90% of the antibody-drug conjugates have a drug loading of 4.

In embodiments, the methods of the present disclosure may be expanded to prepare antibody conjugates with a DAR of 4 using an engineered antibody in which the C220 residue (according to the Eu system of Kabat) of the heavy chain is substituted by another residue, where the method comprises contacting the antibody with a reducing agent selected from the group consisting of 2-[2-(Diphenylphosphino)ethyl]pyridine, 3-(Diphenylphosphino)benzenesulfonic acid, 4-(Diphenylphosphino)benzoic acid, 2-(Diphenylphosphino)ethylamine, 3-(diphenylphosphino)propylamine, 3-(Diphenylphosphino)propionic acid, 2-(diisopropylphosphino)ethylamine, and salts thereof. In embodiments, the reducing agent is selected from the group consisting of 3-(Diphenylphosphino)benzenesulfonic acid, 4-(Diphenylphosphino)benzoic acid, 2-(Diphenylphosphino)ethylamine, 3-(diphenylphosphino)propylamine, 3-(Diphenylphosphino)propionic acid, and salts thereof. In embodiments, the reducing agent is selected from the group consisting of 4-(Diphenylphosphino)benzoic acid, 2-(Diphenylphosphino)ethylamine, 3-(diphenylphosphino)propylamine, 3-(Diphenylphosphino)propionic acid, and salts thereof. In embodiments, the reducing agent is selected from the group consisting of 4-(Diphenylphosphino)benzoic acid, 2-(Diphenylphosphino)ethylamine, 3-(diphenylphosphino)propylamine, and salts thereof. The two cysteines at LC214 (C6v2) that are revealed by the selective reduction process according to the present disclosure may then be capped (by, for example, contacting the antibody with N-ethylmaleimide or another maleimide containing small molecule). The method then further involves reducing the two interchain disulfide bonds of the Fc region with TCEP, thus enabling subsequent conjugation of four drug/toxins to the antibody to generate DAR4 species. In embodiments, more than about 70% of the antibody-drug conjugates produced according to the process have a drug loading of 4. In embodiments, more than about 75% of the antibody-drug conjugates have a drug loading of 4. In embodiments, more than about 80% of the antibody-drug conjugates have a drug loading of 4. In embodiments, more than about 85% of the antibody drug-conjugates have a drug loading of 4. In embodiments, more than about 90% of the antibody-drug conjugates have a drug loading of 4.

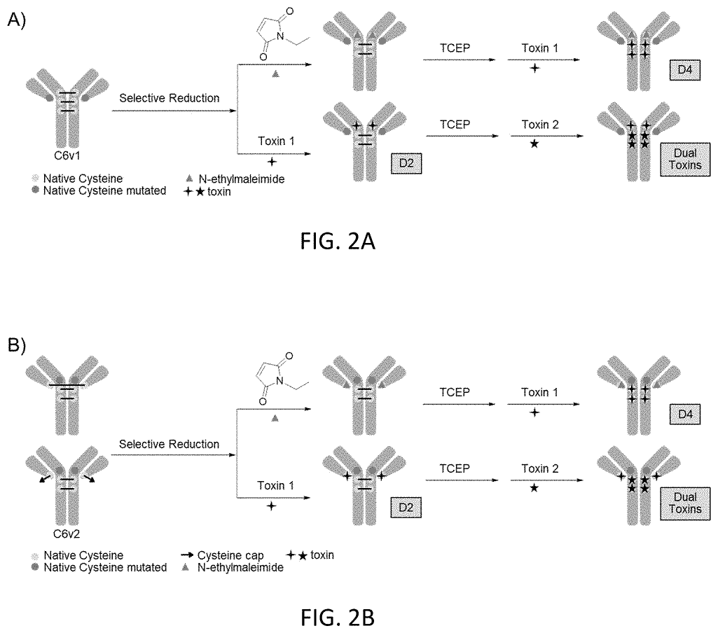

In embodiments, the methods of the present disclosure may be used to prepare antibody-drug conjugates with dual drug-linkers (i.e., two different drug-linkers) in specific positions. In embodiments, the method comprises providing an antibody, and contacting the antibody with a reductant selected from the group consisting of 2-[2-(Diphenylphosphino)ethyl]pyridine, 3-(Diphenylphosphino)benzenesulfonic acid, 4-(Diphenylphosphino)benzoic acid, 2-(Diphenylphosphino)ethylamine, 3-(diphenylphosphino)propylamine, 3-(Diphenylphosphino)propionic acid, 2-(diisopropylphosphino)ethylamine, 2-(diphenylphosphino)benzoic acid, (2-hydroxyphenyl)diphenylphosphine, and salts thereof. In embodiments, the antibody may be an engineered antibody in which the C214 residue of the light chain is substituted by another residue, or an engineered antibody in which the C220 residue of the heavy chain is substituted by another residue. The resulting reduced antibody is then contacted with a first drug-linker compound under conditions sufficient to form a first antibody-drug conjugate. The method then further comprises an additional step of contacting the antibody drug conjugate (i.e., the antibody conjugated to drug/toxin 1) with TCEP to reduce the two inter-chain disulfide bonds at the Fc region, thereby enabling a subsequent conjugation with four of a second drug-linker compound. The resulting antibody conjugate has dual toxins/drugs (i.e., the antibody is conjugated to at least one drug/toxin 1 moiety and at least one drug/toxin 2 moiety) at specific positions, as shown in FIG. 2A-B. In embodiments, more than about 70% of the antibody-drug conjugates produced according to the process have a toxin 1 drug loading of 2 and a toxin 2 drug loading of 4. In embodiments, more than about 75% of the antibody-drug conjugates have a toxin 1 drug loading of 2 and a toxin 2 drug loading of 4. In embodiments, more than about 80% of the antibody-drug conjugates have a toxin 1 drug loading of 2 and a toxin 2 drug loading of 4. In embodiments, more than about 85% of the antibody drug-conjugates have a toxin 1 drug loading of 2 and a toxin 2 drug loading of 4. In embodiments, more than about 90% of the antibody-drug conjugates have a toxin 1 drug loading of 2 and a toxin 2 drug loading of 4.

As shown in FIG. 2A, the methods of the present disclosure may be used to prepare antibody-drug conjugates with dual drug-linkers (i.e., two different drug-linkers) in specific positions using an engineered cysteine antibody in which the C214 residue (according to the Eu system of Kabat) of the light chain is substituted by another residue, where the method involves contacting the antibody with a reducing agent selected from the group consisting of: 2-[2-(Diphenylphosphino)ethyl]pyridine, 3-(Diphenylphosphino)benzenesulfonic acid, 4-(Diphenylphosphino)benzoic acid, 2-(Diphenylphosphino)ethylamine, 3-(diphenylphosphino)propylamine, 2-(diphenylphosphino)benzoic acid, (2-hydroxyphenyl)diphenylphosphine, and salts thereof. In embodiments, the reducing agent is selected from the group consisting of 3-(Diphenylphosphino)benzenesulfonic acid, 4-(Diphenylphosphino)benzoic acid, 2-(Diphenylphosphino)ethylamine, 3-(diphenylphosphino)propylamine, 2-(diphenylphosphino)benzoic acid, (2-hydroxyphenyl)diphenylphosphine, and salts thereof. In embodiments, the reducing agent is selected from the group consisting of 3-(Diphenylphosphino)benzenesulfonic acid, 4-(Diphenylphosphino)benzoic acid, 2-(Diphenylphosphino)ethylamine, 2-(diphenylphosphino)benzoic acid, and salts thereof. The resulting reduced antibody is then contacted with a first drug-linker compound under conditions sufficient to form an antibody-drug conjugate. The method then further comprises an additional step of contacting the first antibody-drug conjugate (i.e., the antibody conjugated to the drug/toxin 1) with TCEP to reduce the two inter-chain disulfide bonds at the Fc region, thereby enabling a subsequent conjugation with four of a second drug-linker compound. The resulting antibody conjugate has dual toxins/drugs (i.e., the antibody is conjugated to at least one drug/toxin 1 moiety and at least one drug/toxin 2 moiety) at specific positions, as shown in FIG. 2A. In embodiments, more than about 70% of the antibody-drug conjugates produced according to the process have a toxin 1 drug loading of 2 and a toxin 2 drug loading of 4. In embodiments, more than about 75% of the antibody-drug conjugates have a toxin 1 drug loading of 2 and a toxin 2 drug loading of 4. In embodiments, more than about 80% of the antibody-drug conjugates have a toxin 1 drug loading of 2 and a toxin 2 drug loading of 4. In embodiments, more than about 85% of the antibody drug-conjugates have a toxin 1 drug loading of 2 and a toxin 2 drug loading of 4. In embodiments, more than about 90% of the antibody-drug conjugates have a toxin 1 drug loading of 2 and a toxin 2 drug loading of 4.

As shown in FIG. 2B, the methods of the present disclosure may be used to prepare antibody-drug conjugates with dual drug-linkers (i.e., two different drug-linkers) in specific positions using an engineered cysteine antibody in which the C220 residue (according to the Eu system of Kabat) of the heavy chain is substituted by another residue, where the method involves contacting the antibody with a reducing agent selected from the group consisting of: 2-[2-(Diphenylphosphino)ethyl]pyridine, 3-(Diphenylphosphino)benzenesulfonic acid, 4-(Diphenylphosphino)benzoic acid, 2-(Diphenylphosphino)ethylamine, 3-(diphenylphosphino)propylamine, 3-(Diphenylphosphino)propionic acid, 2-(diisopropylphosphino)ethylamine, and salts thereof. In embodiments, the reducing agent is selected from the group consisting of 3-(Diphenylphosphino)benzenesulfonic acid, 4-(Diphenylphosphino)benzoic acid, 2-(Diphenylphosphino)ethylamine, 3-(diphenylphosphino)propylamine, 3-(Diphenylphosphino)propionic acid, and salts thereof. In embodiments, the reducing agent is selected from the group consisting of 4-(Diphenylphosphino)benzoic acid, 2-(Diphenylphosphino)ethylamine, 3-(diphenylphosphino)propylamine, 3-(Diphenylphosphino)propionic acid, and salts thereof. In embodiments, the reducing agent is selected from the group consisting of 4-(Diphenylphosphino)benzoic acid, 2-(Diphenylphosphino)ethylamine, 3-(diphenylphosphino)propylamine, and salts thereof. The resulting reduced antibody is then contacted with a first drug-linker compound under conditions sufficient to form an antibody-drug conjugate. The method then further comprises an additional step of contacting the first antibody-drug conjugate (i.e., the antibody conjugated to the drug/toxin 1) with TCEP to reduce the two inter-chain disulfide bonds at the Fc region, thereby enabling a subsequent conjugation with four of a second type of drug/toxin (drug/toxin 2). The resulting antibody conjugate has dual toxins/drugs (i.e., the antibody is conjugated to at least one drug/toxin 1 moiety and at least one drug/toxin 2 moiety) at specific positions, as shown in FIG. 2B. In embodiments, more than about 70% of the antibody-drug conjugates produced according to the process have a toxin 1 drug loading of 2 and a toxin 2 drug loading of 4. In embodiments, more than about 75% of the antibody-drug conjugates have a toxin 1 drug loading of 2 and a toxin 2 drug loading of 4. In embodiments, more than about 80% of the antibody-drug conjugates have a toxin 1 drug loading of 2 and a toxin 2 drug loading of 4. In embodiments, more than about 85% of the antibody drug-conjugates have a toxin 1 drug loading of 2 and a toxin 2 drug loading of 4. In embodiments, more than about 90% of the antibody-drug conjugates have a toxin 1 drug loading of 2 and a toxin 2 drug loading of 4.

In embodiments, the processes of the present disclosure described above may be carried out under mild conditions. In embodiments, the reaction temperature may be from about 0° C. to about 20° C., or about 10° C. In embodiments, the temperature is from about 0° C. to about 4° C. In embodiments, the temperature is about 0° C. In embodiments, the pH is from about 5 to about 9, or from about 6 to about 8.

In embodiments, the processes according to the present disclosure may be performed in a buffered aqueous solution. Suitable buffers include, for example, phosphate-buffered saline buffered aqueous solution, citrate buffered aqueous solution, histidine buffered aqueous solution, tris buffered aqueous solution, gluconate buffered aqueous solution, adipic acid buffered aqueous solution, lactic acid buffered aqueous solution, acetate buffered aqueous solution, or succinate buffered aqueous solution.

### EXAMPLES

### Example 1

### Selective Reduction of C6v1 and C6v2 Antibodies with Phosphine-Based Reductants

Unlike traditional engineered cysteine antibodies in which the newly introduced cysteine residues are commonly capped with a mixture of non-encoded cysteine molecules (e.g., cysteine or glutathione), cysteine deletion mutants C6v1 and C6v2 form unpaired cysteines at their native positions, e.g., HC220 for C6v1 and LC214 for C6v2. By Reverse-Phase HPLC (RP-HPLC) and LCMS characterization, it was determined that the two cysteines at HC220 of C6v1 form disulfide bond, while the two cysteines at LC214 of C6v2 also mostly form disulfide bond, with a varying percentage (8-35%) that are capped, as shown in FIGS. 1A and 1B.

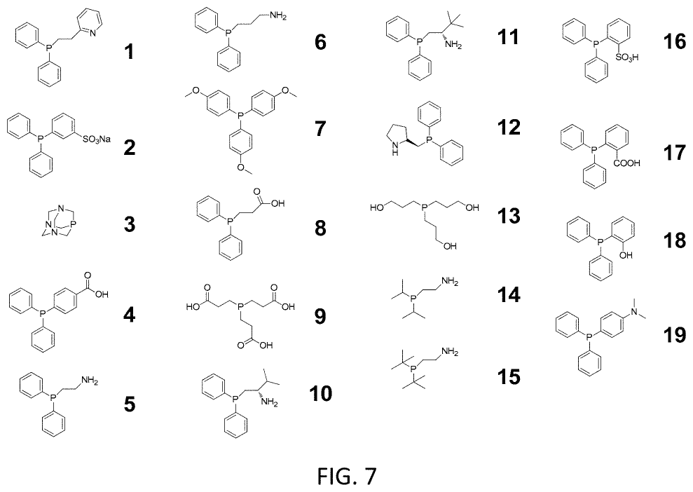

Nineteen phosphine-based reductants obtained from Sigma Aldrich and Strem Chemicals were analyzed for their ability to selectively reduce the unpaired cysteines of C6v1 and C6v2. For purposes of these examples, the cysteine at the HC220 position for C6v2 and the cysteine at the LC214 position for C6v1 was replaced by alanine. The nineteen reductants are listed below in Table 1 and illustrated in FIG. 7.

TABLE 1
Reductants

| Reductant | | |
|---|---|---|
| No. | Reductant name | Structure |
| 1 | 2-[2-(Diphenylphosphino)ethyl]pyridine | 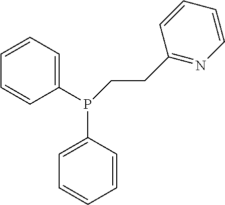 [SMILES: c1ccc(P(CCc2ccccn2)c2ccccc2)cc1] |
| 2 | 3-(Diphenylphosphino)benzenesulfonic acid sodium salt | 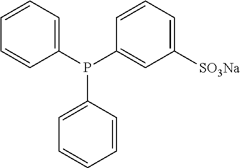 |
| 3 | 1,3,5-triaza-7-phosphatricyclo- [3.3.1.13,7]decane (PTA) | 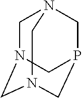 |
| 4 | 4-(Diphenylphosphino)benzoic acid | 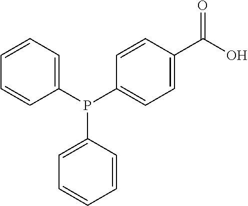 [SMILES: O=C(O)c1ccc(P(c2ccccc2)c2ccccc2)cc1] |
| 5 | 2-(Diphenylphosphino)ethylamine | 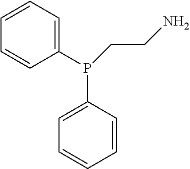 [SMILES: NCCP(c1ccccc1)c1ccccc1] |
| 6 | 3-(diphenylphosphino)propylamine | 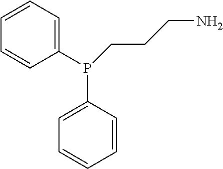 [SMILES: NCCCP(c1ccccc1)c1ccccc1] |
| 7 | Tris(4-methoxyphenyl)phosphine | 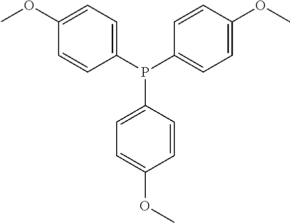 |
| 8 | 3-(Diphenylphosphino)propionic acid | 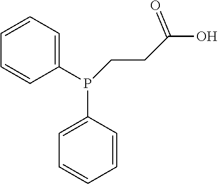 [SMILES: O=C(O)CCP(c1ccccc1)c1ccccc1] |
| 9 | Tris(2-carboxyethyl)phosphine (TCEP) | 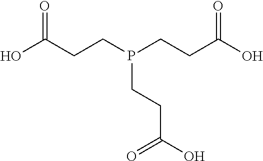 [SMILES: O=C(O)CCP(CCC(=O)O)CCC(=O)O] |
| 10 | (S)-1-(Diphenylphosphino)-3-methyl-2- butylamine | 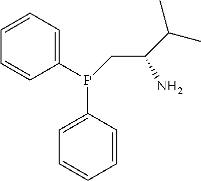 [SMILES: CC(C)[C@H](N)CP(c1ccccc1)c1ccccc1] |
| 11 | (S)-1-(Diphenylphosphino)-3,3- dimethylbutan-2-amine | 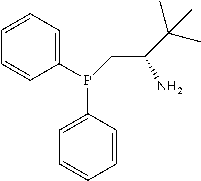 [SMILES: CC(C)(C)[C@H](N)CP(c1ccccc1)c1ccccc1] |
| 12 | (r)-2- ((diphenylphosphino)methyl)pyrrolidine | 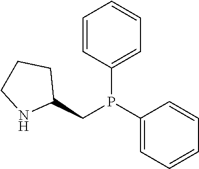 |
| 13 | Tris(hydroxypropyl)phosphine | 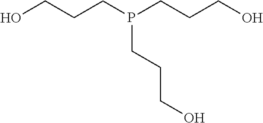 [SMILES: OCCCP(CCCO)CCCO] |
| 14 | 2-(diisopropylphosphino)ethylamine | 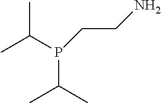 |
| 15 | 2-(di-tert-butylphosphino)ethylamine | 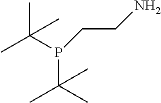 |
| 16 | 2-(diphenylphosphino)benzenesulfonic acid | 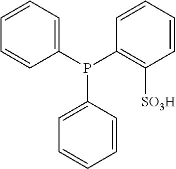 [SMILES: O=S(=O)(O)c1ccccc1P(c1ccccc1)c1ccccc1] |
| 17 | 2-(diphenylphosphino)benzoic acid | 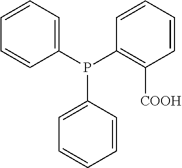 [SMILES: O=C(O)c1ccccc1P(c1ccccc1)c1ccccc1] |
| 18 | (2-hydroxyphenyl)diphenylphosphine | 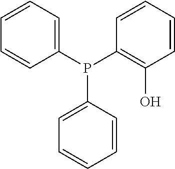 [SMILES: Oc1ccccc1P(c1ccccc1)c1ccccc1] |
| 19 | 4-(diphenylphosphino)-N,N- dimethylaniline | 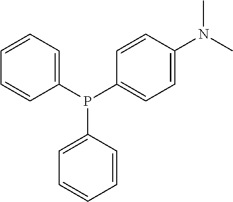 [SMILES: CN(C)c1ccc(P(c2ccccc2)c2ccccc2)cc1] |

The reductants were prepared as 10-20 mM solution in dimethylformamide (DMF) and diluted with water to 5-10 mM. The C6v1 and C6v2 of Cetuximab antibodies in Dulbecco's phosphate-buffered saline (DPBS) buffer pH 7.2 were used for screening. The EGFR targeting antibody Cetuximab is described in WO 2009/134776, the entire disclosure of which is incorporated by reference herein in its entirety. The antibodies were mixed with 2-4 equivalents of the reductants and incubated at 0° C. overnight (16-20 hours). MC-VC-MMAE was then added to the solution for conjugation, followed by hydrophobic interaction chromatography (HIC) analysis of DAR profile. The peak area % of desired DAR2 species having two toxins conjugated to the antibody is listed in Table 2.

TABLE 2
Yield of D2 by HIC chromatographic analysis in reactions
of Cetuximab-C6v1 and Cetuximab-C6v2 with phosphine-based
reductants 1-19 followed by conjugation with MC-VC-MMAE

| | C6v1 | C6v2 |
|---|---|---|
| REDUCTANT | DAR2 YIELD (%) | DAR2 YIELD (%) |
| 9 (TCEP) | 57.8 | 51.3 |
| 3, 15, 16 | <10 | <10 |
| 10, 11, 12, 13 | 10-50 | 10-50 |
| 7, 19 | 10-50 | <10 |
| 1 | 64.1 | 72.4 |
| 2 | 87.5 | 77.9 |
| 4 | 86.4 | 88.9 |
| 5 | 84.9 | 87.1 |
| 6 | 72.1 | 85.4 |
| 8 | 52.2 | 74.6 |
| 14 | 54.4 | 70.3 |
| 17 | 83.3 | 30.7 |
| 18 | 77.6 | 54.4 |
| TCEP/DHAA | 62.1 | N/A |

As shown in Table 2, among the reductants screened under the conditions described above, reductants 2, 4, 5, and 17 produce >80% of DAR2 for C6v1, and reductants 4, 5, and 6 produce >85% of DAR2 for C6v2. The relativities of the reductants vary for C6v1 and C6v2 mutants, indicating the different sensitivity of these two antibodies to the physiochemical property of the reductants.

TCEP (reductant 9) shows poor selectivity towards C6v1 and C6v2 antibodies, and non-selectively gives a mixture of drug loading species with only 58% and 51% of DAR2 generated. This is also seen with other strong reductants (such as formulas 13 and 14) where significantly more reduction of the inter-chain disulfide bonds at the Fc region were observed, as shown by the high drug loading species by HIC analysis.

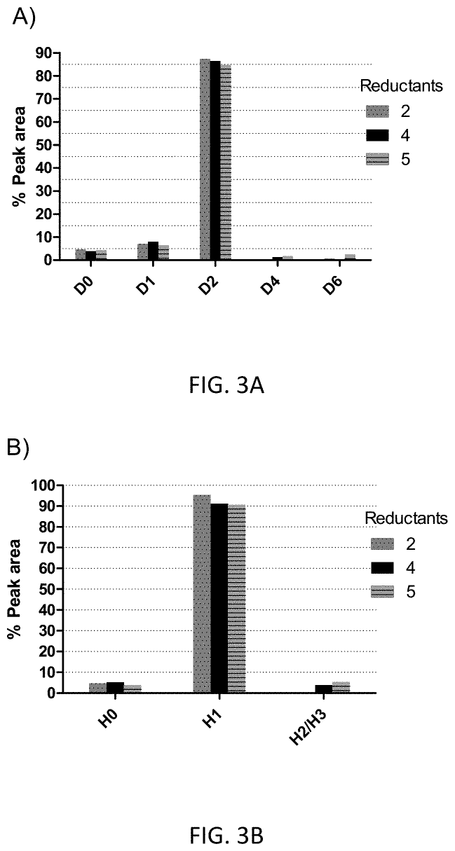

Reductants 2, 4, and 5 result in ≥85%, of DAR2 for C6v1 and show not only a high level of desired DAR2 or D2 by HIC, but also a very low level of high DAR species (DAR4/DAR6 or D4/D6), indicating high selectivity of the reductants, as shown in FIG. 3A. The presence of DAR0/DAR1 or D0/D1 is typical for engineered cysteine antibodies. FIG. 3B shows the analysis by RP-HPLC, where H0 indicates heavy chain with no toxin conjugated, H1 indicates heavy chain with one toxin conjugated, and H2/H3 indicates heavy chain with two or three toxins conjugated. The data shows >90% of H1. Minimal reduction of the inter-chain disulfide bonds at the Fc region is observed as indicated by the low level of H2/H3. The reductant 2 shows the best profile for C6v1, where close to 90% of D2 and only <0.8% of D4/D6 is observed by HIC. By RP-HPLC, the desired H1 is >95% and the undesirable H2/H3 is negligible.

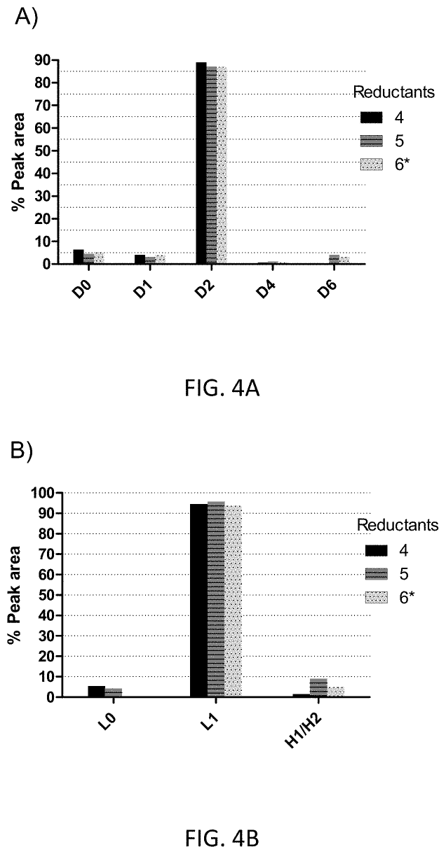

Reductants 4, 5, and 6 not only result in >85% of DAR2 for C6v2, but also produce a low level of the high drug loading species (D4/D6), as shown in FIG. 4A. FIG. 4B shows the analysis by RP-HPLC, where L0 and L1 represent no toxin or one toxin conjugation at the LC214 position, respectively. Their percentages are calculated as L0%=(L0/(L0+L1))% and L1%=(L1/(L0+L1))%. H1/H2 represents the heavy chain with one or two toxins conjugated and is the indication of undesirable reduction of interchain disulfide bonds in the Fc region. The data shows >93% of L1. The undesirable H1/H2 is controlled to low levels. Reductant 4 shows the best selectivity for C6v2, where close to 90% of D2 and only <0.7% of D4/D6 is observed by HIC. By RP-HPLC, the desired L1 is close to 95% and the undesirable H1/H2 is only 1.5%.

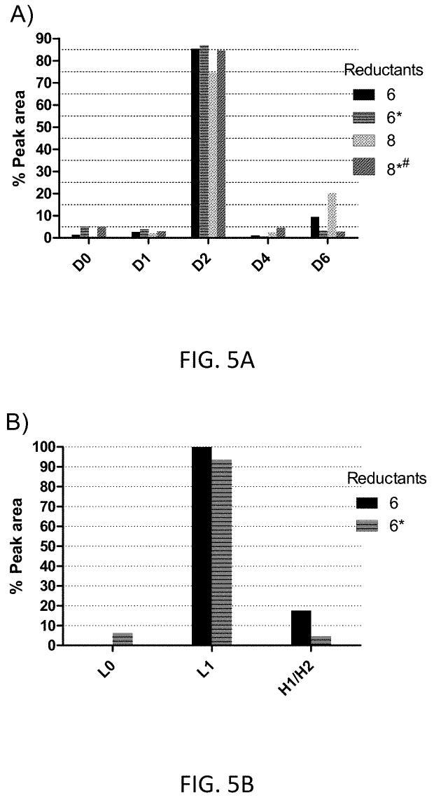

Some reductants exhibit some sensitivity towards buffer pH during reduction of C6v2. For example, reductant 6 produces a majority of DAR2 in DPBS pH 7.2 buffer but contains relatively high amount of D4/D6 species. When the pH is raised to 8, the level of high drug loading species is controlled to less than 4% while keeping similar levels of DAR2 species (>85%), as shown in FIG. 5A. This is also shown by RP-HPLC where the H1/H2 is greatly reduced, indicating increased selectivity at higher pH, as shown in FIG. 5B. Buffer pH has a larger impact on reductant 8 when the buffer pH is raised from 7.2 to 8, as the DAR2 species increases from 75% to 85% by controlling the undesirable inter-chain disulfide bond reduction (D4/D6).

### Example 2

### Selective Reduction of Additional C6v1 and C6v2 Antibodies

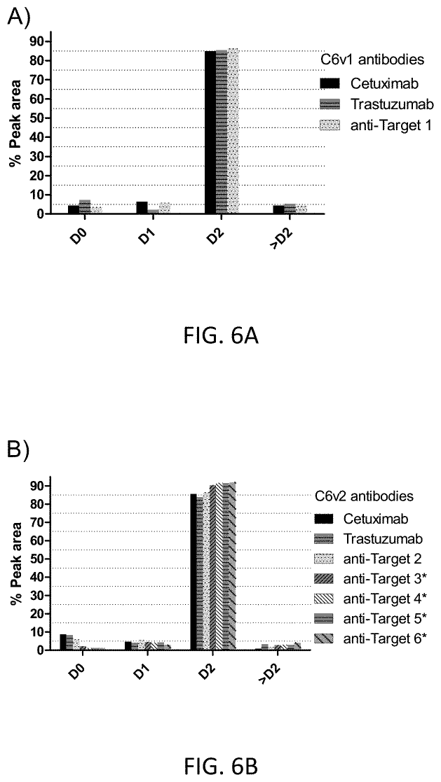

The reduction process was also evaluated on several additional C6v1 and C6v2 antibodies that have different sequences and are designed for various targets or no targets. Three C6v1 mutant antibodies (Cetuximab-C6v1, Trastuzumab-C6v1, and anti-Target 1-c6v1) tested with reductant 5, 2-(Diphenylphosphino)ethylamine, all show similar HIC profiles with ≥85%, and ≤5%, of high drug loading (>D2) species, as shown in FIG. 6A. For C6v2 antibodies, with reductant 4, all seven antibodies (Cetuximab-C6v2, Trastuzumab-C6v2, anti-Target 2-C6v2, anti-Target 3-C6v2, anti-Target 4-C6v2, anti-Target 5-C6v2, and anti-Target 6-C6v2) show ≥85% D2 and <4% of high drug loading (>D2) species by HIC when reduced with 4-(Diphenylphosphino)benzoic acid (reductant 4), despite their difference in sequence and the percentage (8-35%) of capped cysteine at position LC214, as shown in FIG. 6B. The data show the robustness of selective reduction of these reductants towards C6v1 and C6v2 antibodies, irrespective of the heavy and light chain sequences.

### Example 3

### Application of C6v1 and C6v2 Antibodies for Generation of D4 and Dual Toxin ADCs

In addition to preparing D2 defined ADCs as discussed above, the methods of the present disclosure may be used to prepare D4 defined ADCs, or ADCs with two different drug-linkers (dual toxin ADCs) at specific positions. In the case of the C6v1 antibody, as shown in FIG. 2A, to prepare D4 controlled ADCs, N-ethylmaleimide or other maleimide containing small molecules can be used to cap the two cysteines at HC220 that are selectively revealed by reductant 2, 4, or 5. Further reduction with TCEP of the two-interchain disulfide bonds at the Fc region would enable a subsequent conjugation of four toxins to the monoclonal antibody to generate D4. To prepare ADCs with dual toxins, after conjugating one type of drug-linker at the two cysteines of the Fab region of heavy chains that are selectively reduced by reductant 2, 4, or 5, a second step of treatment with TCEP to reduce the two inter-chain disulfide bonds at the Fc region would enable a subsequent conjugation with four of a second type of drug-linker, resulting in an antibody conjugate with dual drug-linkers (i.e., two different types of drug-linkers) at specific positions.

Cetuximab-C6v1 was used to prepare a DAR4 ADC according to this process. Cetuximab-C6v1 was reduced with 2-(Diphenylphosphino)ethylamine (reductant 5). The resulting reduced antibody was treated with N-ethylmaleimide to cap the cysteines revealed through the reduction. The antibody was then reduced with TCEP and conjugated with a drug-linker (toxin 1). The resulting product contained 96.3% D4 as determined by HIC. This process was repeated using a different drug-linker (toxin 2), and yielded 93.0% D4 as determined by HIC.

Cetuximab-C6v1 was used to prepare a dual-toxin ADC according to this process. Cetuximab-C6v1 was reduced with 2-(Diphenylphosphino)ethylamine (reductant 5), and conjugated with a first drug-linker (toxin 1). The resulting ADC was then treated with TCEP and conjugated with a second drug-linker (toxin 2) to yield a dual-toxin ADC. The purity of the product (product %) as determined by HIC for an ADC with a toxin 1 drug loading of 2 and a toxin 2 drug loading of 4 was 71.2%.

This process was then repeated, except that following reduction with 2-(Diphenylphosphino)ethylamine (reductant 5), the antibody was first conjugated with toxin 2. The resulting ADC was then treated with TCEP and conjugated with toxin 1 to yield a dual-toxin ADC. The purity of the product (product %) as determined by HIC for an ADC with a toxin 1 drug loading of 2 and a toxin 2 drug loading of 4 was 93.5%.

### Example 4

### Selective Reduction of Wild Type Antibodies

The reduction process was also evaluated with respect to a wild type antibody. Reductants 5, 6, 8, and 9 were prepared as above, as 10-20 mM solution in N,N-Dimethylacetamide (DMA) and diluted with water to 5-10 mM. A wild type antibody diluted in PBS/EDTA buffer pH 7.2 was used. The antibody was mixed with 2-3 equivalents of a respective reductant (in particular, reductants 5, 6, 8, and 9) and incubated at 0° C. overnight (16-24 hours) or at 4° C. overnight (for Reductant 9). A linker-drug was added to the solution for conjugation, followed by hydrophobic interaction chromatography (HIC) analysis of the DAR profile. The peak area % of DAR4 species having four linker-drugs conjugated to the antibody is listed in Table 3.

TABLE 3
Yield of D4 by HIC chromatographic analysis in reactions of a wild-type antibody
with phosphine-based reductants followed by conjugation with a toxin

| | 2-(diphenylphosphino)- | 3-(diphenylphosphino)- | 3-(diphenylphosphino)- | TCEP | TCEP |
|---|---|---|---|---|---|
| | ethylamine | propylamine | propionic acid | Reductant | Reductant |
| Reductant | Reductant 5 | Reductant 6 | Reductant 8 | 9 | 9 |
| Equivalents | 3 | 2.2 | 2.2 | 2.2 | 2.2 |
| Temperature | 0° C. | 0° C. | 0° C. | 4° C. | 0° C. |
| % D4 | 57.61 | 53.13 | 55.45 | 48.1 | 45.7 |

Reductants 5, 6, and 8 each were more effective than TCEP at enhancing the yield of the DAR4 species generated from a wild-type antibody.

It is understood that the foregoing detailed description and accompanying examples are merely illustrative and are not to be taken as limitations upon the scope of the present disclosure, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the present disclosure, may be made without departing from the spirit and scope thereof. All publications, patents, and patent applications cited herein are hereby incorporated by reference in their entirety for all purposes.

1. A method for selectively reducing a protein, the method comprising:
contacting the protein with a reducing agent selected from the group consisting of
2-[2-(Diphenylphosphino)ethyl]pyridine;
3-(Diphenylphosphino)benzenesulfonic acid;
4-(Diphenylphosphino)benzoic acid;
2-(Diphenylphosphino)ethylamine;
3-(diphenylphosphino)propylamine;
3-(Diphenylphosphino)propionic acid;
2-(diisopropylphosphino)ethylamine;
2-(diphenylphosphino)benzoic acid;
(2-hydroxyphenyl)diphenylphosphine;
and salts thereof.

2. The method according to claim 1, wherein the protein is an antibody.

3. The method according to claim 2, wherein the antibody comprises at least one cysteine residue that has been deleted or replaced by another amino acid.

4. The method according to claim 3, wherein the cysteine residue has been replaced by another amino acid in the C214 position in the light chain or the C220 position in the heavy chain according to the Eu system of Kabat.

5. The method according to claim 3 or 4, wherein the cysteine residue has been replaced by serine or alanine.

6. The method according to claim 3-5, wherein the cysteine residue in the C214 position in the light chain has been replaced by another amino acid, and wherein the reducing agent is selected from the group consisting of:
2-[2-(Diphenylphosphino)ethyl]pyridine;
3-(Diphenylphosphino)benzenesulfonic acid;
4-(Diphenylphosphino)benzoic acid;
2-(Diphenylphosphino)ethylamine;
3-(diphenylphosphino)propylamine;
2-(diphenylphosphino)benzoic acid;
(2-hydroxyphenyl)diphenylphosphine;
and salts thereof.

7. The method according to claim 3-5, wherein the cysteine residue in the C220 position in the heavy chain has been replaced by another amino acid, and wherein the reducing agent is selected from the group consisting of:
2-[2-(Diphenylphosphino)ethyl]pyridine;
3-(Diphenylphosphino)benzenesulfonic acid;
4-(Diphenylphosphino)benzoic acid;
2-(Diphenylphosphino)ethylamine;
3-(diphenylphosphino)propylamine;
3-(Diphenylphosphino)propionic acid;
2-(diisopropylphosphino)ethylamine;
and salts thereof.

8. A method for preparing an antibody-drug conjugate, the method comprising:
contacting an antibody with a reducing agent selected from the group consisting of:
2-[2-(Diphenylphosphino)ethyl]pyridine;
3-(Diphenylphosphino)benzenesulfonic acid;
4-(Diphenylphosphino)benzoic acid;
2-(Diphenylphosphino)ethylamine;
3-(diphenylphosphino)propylamine;
3-(Diphenylphosphino)propionic acid;
2-(diisopropylphosphino)ethylamine;
2-(diphenylphosphino)benzoic acid;
(2-hydroxyphenyl)diphenylphosphine;
and salts thereof; and
combining the antibody with a drug-linker compound under conditions sufficient to form an antibody-drug conjugate.

9. The method according to claim 8, wherein more than about 70% of the antibody-drug conjugates produced according to the process have a drug loading of 2.

10. The method according to claim 8, wherein more than about 80% of the antibody-drug conjugates produced according to the process have a drug loading of 2.

11. A method for the preparation of an antibody conjugate, the method comprising:
providing an antibody, wherein a cysteine residue of the antibody has been replaced by another amino acid in the C214 position in the light chain or the C220 position in the heavy chain according to the Eu system of Kabat, and
contacting the antibody with a reducing agent selected from the group consisting of
2-[2-(Diphenylphosphino)ethyl]pyridine;
3-(Diphenylphosphino)benzenesulfonic acid;
4-(Diphenylphosphino)benzoic acid;
2-(Diphenylphosphino)ethylamine;
3-(diphenylphosphino)propylamine;
3-(Diphenylphosphino)propionic acid;
2-(diisopropylphosphino)ethylamine;
2-(diphenylphosphino)benzoic acid;
(2-hydroxyphenyl)diphenylphosphine;
and salts thereof.

12. The method according to claim 11, further comprising conjugating the reduced antibody to a first toxin or therapeutic moiety to yield a first antibody conjugate.

13. The method according to claim 12, comprising reducing the first antibody conjugate with TCEP to yield a reduced first antibody conjugate.

14. The method according to claim 13, comprising conjugating the reduced first antibody conjugate to a second toxin or therapeutic moiety to yield a dual drug-linker antibody drug conjugate.

15. The method according to claim 11, comprising reacting the reduced antibody with a maleimide containing compound to cap the cysteines revealed when the antibody is contacted with the reducing agent; and subsequently further reducing the antibody with TCEP to reduce at least two inter-chain disulfide bonds in the Fc region.

16. The method according to claim 15, further comprising conjugating at least four drug-linkers or therapeutic moieties to the antibody.

17. The method according to claim 16, wherein more than about 70% of the antibody-drug conjugates prepared according to the process have a drug loading of 4.

18. The method according to claim 16, wherein more than about 80% of the antibody-drug conjugates prepared according to the process have a drug loading of 4.

19. An antibody conjugate prepared according to claim 11.

20. A composition comprising antibody conjugates prepared according to claim 11, wherein the composition has an average DAR of 2.
